(19) United States
(12) Patent Application Publication
(10) Pub. No.: US 2023/0191170 A1
(43) Pub. Date: Jun. 22, 2023

(54) RESPIRATOR MASK WITH INTEGATED ULTRAVIOLET LIGHTING SYSTEM

(71) Applicants: Alex Demitri Smike, Kingston 8 (JM); Nickolas David Smikle, Kingston 8 (JM)

(72) Inventors: Alex Demitri Smike, Kingston 8 (JM); Nickolas David Smikle, Kingston 8 (JM)

(21) Appl. No.: 17/559,428

(22) Filed: Dec. 22, 2021

(51) Int. Cl. A62B 18/02; A62B 18/08; A62B 9/06; A61L 9/20

(52) U.S. Cl. CPC A62B 18/02; A62B 18/084; A62B 9/06; A61L 9/20; A61L 2209/12; A61L 2209/111

(57) ABSTRACT

A respirator mask, having an air intake chamber for taking air into the mask from the outside, an ultraviolet lighting system within the interior of the mask, and a control system for controlling the operation of the lighting system. The lighting system mitigates the health hazards caused by bacteria, germs and viruses within the air within the air intake chamber.

The present invention generally relates to the field of personal safety devices and more particularly, is directed to a respirator mask with an integrated ultraviolet lighting system for mitigating health hazards caused by bacteria, germs and viruses.

### BACKGROUND OF THE INVENTION

Throughout history, the world has experienced many catastrophes and threats to mankind. Some, such as armed conflicts, explosions, major fires and releases of toxic substances into the environment, are man-made. Others are the result of natural causes such as floods, storms, earthquakes, droughts, forest fires and volcanic eruptions.

Epidemics and pandemics have also accounted for many of the world's medical catastrophes. It is estimated that during the 14^{th} century, the Black Plague caused 75 million people to die. The Spanish Flu of 1918 is believed to have afflicted one-third of the world's population and left 50 million dead. More recently, HIV/AIDS has infected at least 60 million people and an estimated 25 million people have died.

Today the world is facing Covid-19, the full breath and long-term dimensions of which are not yet known.

As Covid-19 has demonstrated, the need for personal safety protection, such as masks and hand sanitizers, cannot be over emphasized.

Respirators and surgical masks are examples of personal protective devices that protect wearers from inhaling hazardous airborne particles and from such particles contaminating the face. While such personal protective devices and methods continue to improve, they remain deficient in a number of areas.

Accordingly, the objective of the present invention is to provide an improved respiratory mask.

### SUMMARY OF THE INVENTION

The present invention provides a respirator mask system for wearing on the face that features a pre-filtered intake air chamber with UV-C/Far UV-C radiation source(s), in the form for example, of light emitting diodes (LEDs), that inactivates airborne pathogens in droplet and aerosol form. Airborne pathogens include air born viruses such as coronavirus, influenza virus, rhinovirus, and bacteria. The respirator utilizes UV-C LED technology as its irradiation source to destroy the susceptible virus/bacterial cells. The UV radiation is housed within the mask's air chamber which is fully enclosed to prevent radiation damage to the wearer's skin as well other human bodies in the vicinity of the wearer.

### BRIEF DESCRIPTION OF THE DRAWINGS

The novel features of the present invention are set out with particularity in the appended claims, but the invention will be understood more fully and clearly from the following detailed description of the invention as set forth in the accompanying drawings in which:

FIG. 1 is a side view of one embodiment of a respirator mask in accordance with the present invention;

FIGS. 2 and 3 are front views of one embodiment of a respirator mask in accordance with the present invention;

FIG. 4 is top view of one embodiment of a respirator mask in accordance with the present invention;

FIG. 5 is a cross-sectional side view of one embodiment of a respirator mask in accordance with the present invention;

FIG. 6 is a rear view of one embodiment of a respirator mask in accordance with the present invention;

FIG. 7 is an electrical block diagram of one embodiment of a power supply in accordance with the present invention;

FIG. 8 is an electrical block diagram of one embodiment of a power supply and control system in accordance with the present invention;

FIGS. 9 and 10 are electrical block diagrams of a further embodiment of a control system in accordance with the present invention;

FIG. 11 illustrates one embodiment of an air treatment chamber in accordance with the present invention; and

FIGS. 12 and 13 are flow charts of embodiments of the operation of the control system in accordance with the present invention.

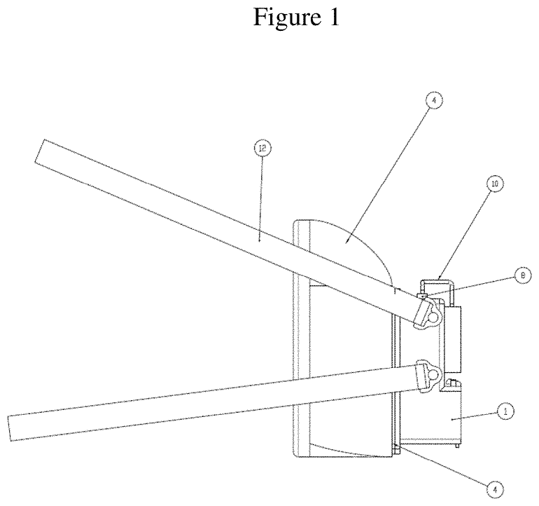

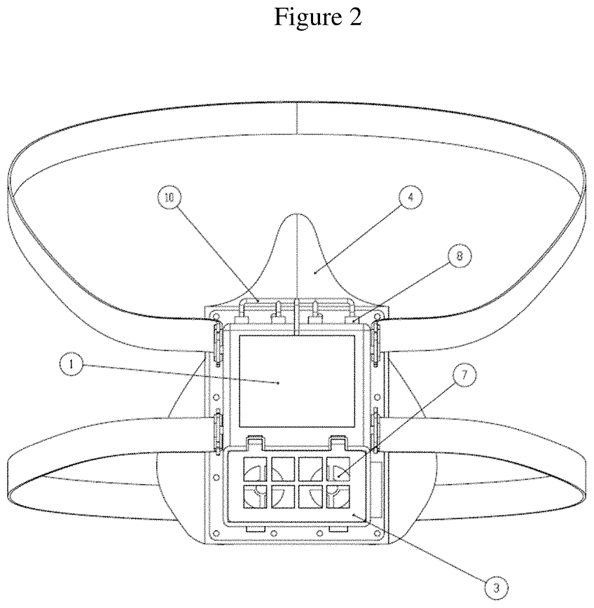

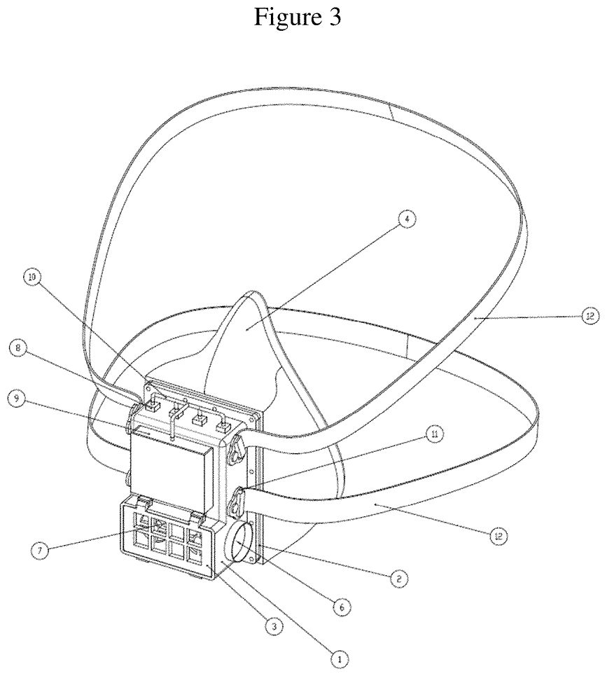

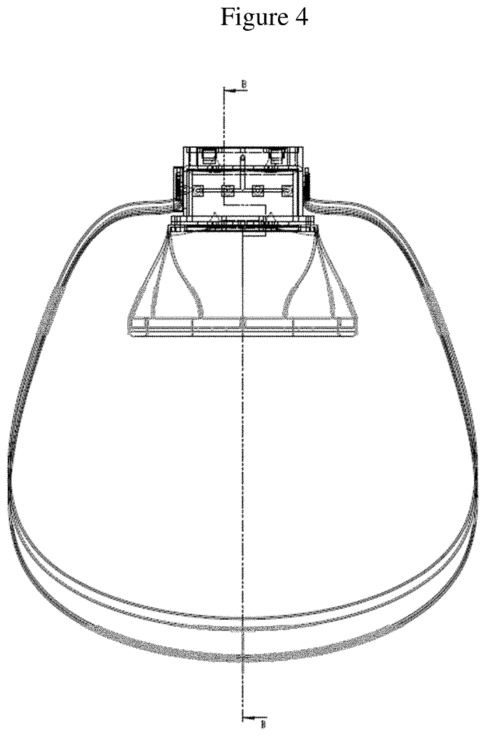

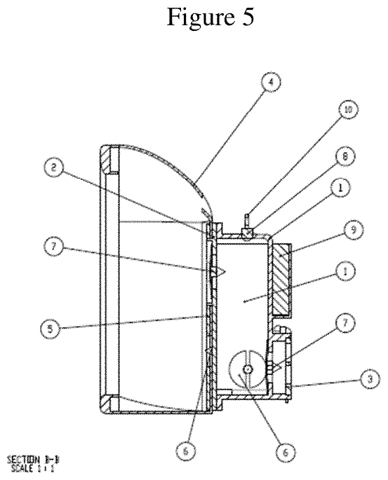

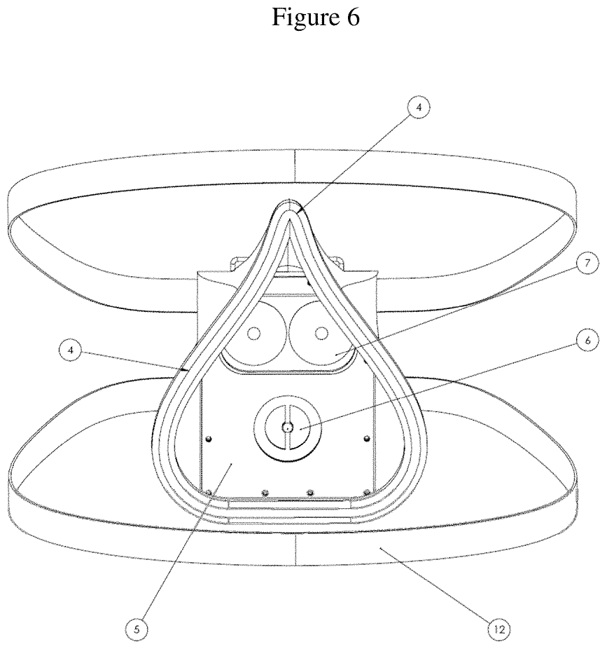

### DESCRIPTION OF THE PREFERRED EMBODIMENT

A preferred embodiment of the present invention will now described with reference to the accompanying drawings.

The LEDs 8 that forms the irradiation source of the present invention are connected in series to an LED driver circuit and a source of electrical power 9, for example, a battery. The batteries and driver circuit are mounted within a modular section of the mask that is sealed off from environmental factors such as heat and water incursion. The battery and LED driver are of a technology that permit lightweight and small packaging. Such a battery could be Lithium Ion battery technology.

The LEDs are inserted within the Air Inactivation Chamber 1 in such a way that they are centrally and symmetrically located at one end of the chamber. The light/irradiation from the LEDs are unobstructed and thus the LEDs are inserted to a depth across the boundary of the chamber to allow the full view angle of the LEDs. The LEDs are sealed internally and externally to prevent water damage and other external factors.

The LEDs will emit UV type C radiation. This radiation is in the range of 230-280 nanometer wavelengths. Far UV-C is of a shorter wavelength and has been demonstrated to be less harmful to exposed mammalian skin. The intensity of the radiation from the LEDs determine what percentage of the total viral count contained within a volume of suspended virus particles will be destroyed over a fixed period. From this, the LEDs' intensity, in milliwatts per centimeter squared will be determined for the respirator to inactivate contaminated air flowing at rates typical of human air consumption for various activity levels. For example, 18,000 mW/cm2 LEDs will be sufficient to inactivate air contaminated with SARS CoV2 virus being inhaled at a rate of 170 liters per second, which is a very rapid inhalation rate.

A respirator equipped with this LED assembly will be able to provide over 24 hours of use with a typical 4,000 mAh Lithium Ion battery found in generic smart phones. A charge port to allow charging of the battery is also present, according to the present invention. This may be separate from the respirator where the batteries are removed and charged without the respirator, or the charging facility may be included with the battery and LED driver module whereby a USB type port is present for charging with a separate charge adapter connected to household voltage.

The respirator body/housing consists of a moulded mouthpiece 4, head straps 12 to affix the mouth piece to the face creating an airtight seal between the wearer's face and the respirator, the Air Inactivation Chamber 9 affixed the frontal portion of the mouthpiece, Intake Air Valves 7 housed in bores connecting the inside volume of the air chamber to the inside volume of the mouth piece, LED port ways where the LEDs 8 are affixed to irradiate the internal volume of the chamber, electronics and energy storage enclosure, prefilter housing, and pre-filter guard 3.

A moulded mouthpiece typical of an airtight respirator is present. This is manufactured from moulded rubber, silicone, or similar type material that is washable with soap and hot water. The mouthpiece has a port to allow the passage of air into and out of the mouthpiece.

Adjustable head straps will be affixed to the air chamber via moulded eyes or hooks 11. The head straps are noosed around the back of the head and tightened to create a sealing pressure between the wearer's face and the mouthpiece of the respirator.

The Air Inactivation Chamber 1 is a straight passage of a closed extruded shape such as rectangular, circular, oval shape, whereby air is allowed to pass all the while exposed to the radiation from the UV-C LED's which are located in the chamber in a manner to expose all the internal volume to radiation, as well as to permit the maximum amount of exposure to any given particle that enters the chamber from the inlet boundary and leaves at the exit boundary. The length and cross-sectional area of the chamber are a function of the LED radiation intensity, the desired survival concentration of the pathogen, the susceptibility of the pathogen to UVC radiation and the flow rate of air passing through the respirator. The air chamber ensures pathogens are exposed long enough to radiation to achieve the desired germicidal effect. For example, it may require 40 milliseconds of exposure for a discrete volume of SARS CoV contaminated air to be inactivated to 1% concentration with 18,000 mW of UV-C. The air intake chamber will be sized in accordance with the maximum expected air flow rate in mind to impart the desired survival rate.

The air inactivation chamber shall be of a rigid impervious washable material such as thermoplastic plastic. It must not be degraded by washing or cleaning. It must also be resistant to UVC or have UVC resistant coatings.

Intake air valves 7 are present to allow disinfected air into the mouthpiece from the inactivation chamber. The valves are flexible and elastic material that only allow airflow in one direction and return to a normally closed state when there is no airflow or flow in the opposite direction.

An outlet air valve 6 will let exhaled air out of the mask. This valve is located in a position where exhaled air must traverse through the inactivation chamber, sanitizing air that may be infected (should the wearer be infected with disease or is incubating the disease) before it is released to the environment.

A pre-filter (not shown but is a replaceable fibrous layer fixed between the filter guard and chamber) is present which is upstream of the inactivation chamber. This is the primary filter which prevents dust particles as well biological molecules which may be infected with pathogens from entering the respirator. The pre-filter is of the typical flat permeable material which is of the same standard used for surgical masks, and other particulate masks and respirators.

The pre-filter is held in place within the respirator housing via the pre-filter guard 3.

This respirator and its filter will meet NIOSH requirements for respirators. It will find its use in the health care sector for health care workers, and any industry or sector where the risk of contracting airborne disease exists for example, public transport, construction, finance, tourism, and travel. The respirator is washable with soap and water and will serve to protect its wearer reliably for years. The batteries are rechargeable, and the electronics are also replaceable at the end of their lifecycle. The respirator will meet or exceed the level of sanitization of existing masks, respirators designed to inhibit the inhalation of infected air and the respirator will also greatly reduce the incidental infection of first responders in healthcare who are exposed to various pathogens daily.

The UV mask's LED power output is regulated by the wearers breathing rate. This results in efficient deactivation of aerosolized pathogens by generating the correct amount of UV radiation proportional to the rate of breathing. LED output will be low when there is steady and slow breathing at low activity levels. At high activity levels with rapid and heavy breathing the LED intensity will increase accordingly to effectively deactivate pathogens suspended in inhaled air.

The mask wearer's breathing rate is measured by a wind sensor. This sensor may be a hotwire anemometer (not shown) which is small enough to be packaged into the mask's housing, and also sensitive enough to detect low wind speeds. Power is applied to the anemometer's sensor to maintain a constant temperature as the breathing rate changes. The speed of the mask wearer's breath is proportional to the heat applied to the sensor to maintain the constant temperature. The anemometer produces a voltage signal that is then received by an LED dimming circuit which is wired to the LED driver to provide varying current to the UV-C LEDS. The LED intensity is proportional to the current, and this varied intensity is mapped to produce the desired inactivation percentage, or pathogen kill rate, for a particular breathing rate.

What is achieved is precise power consumption for varying activity levels and breathing rates resulting in reduced energy consumption, longer battery life, higher energy efficiency and ultimately longer protection for the wearer.

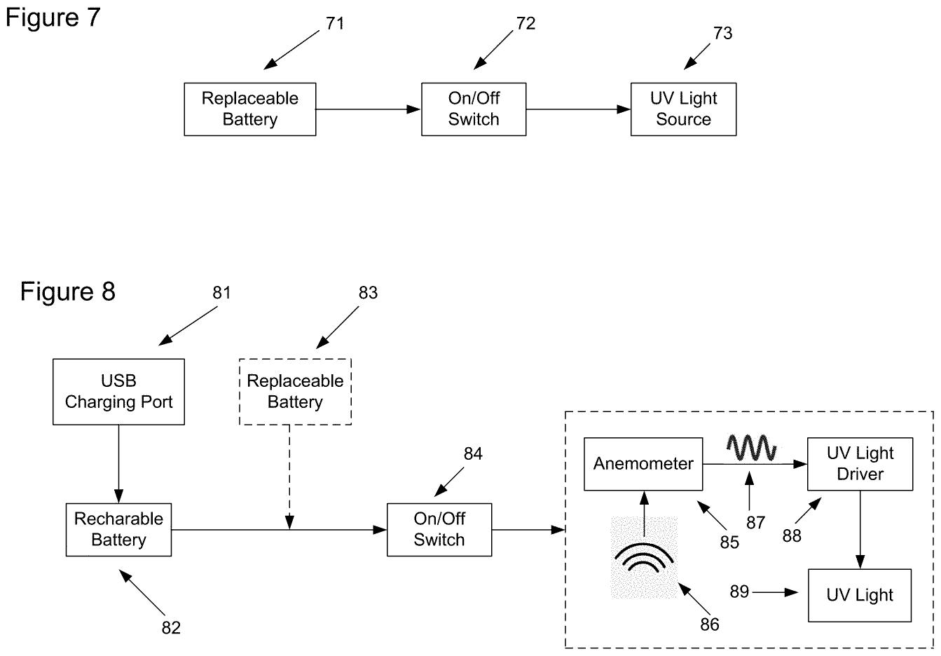

FIGS. 7-13 illustrate embodiments of an electronic control system for the present invention.

As shown in FIG. 7, at its highest level, the system includes a Replaceable Battery 71, and On/Off Switch 72 and the UV Light Source 73.

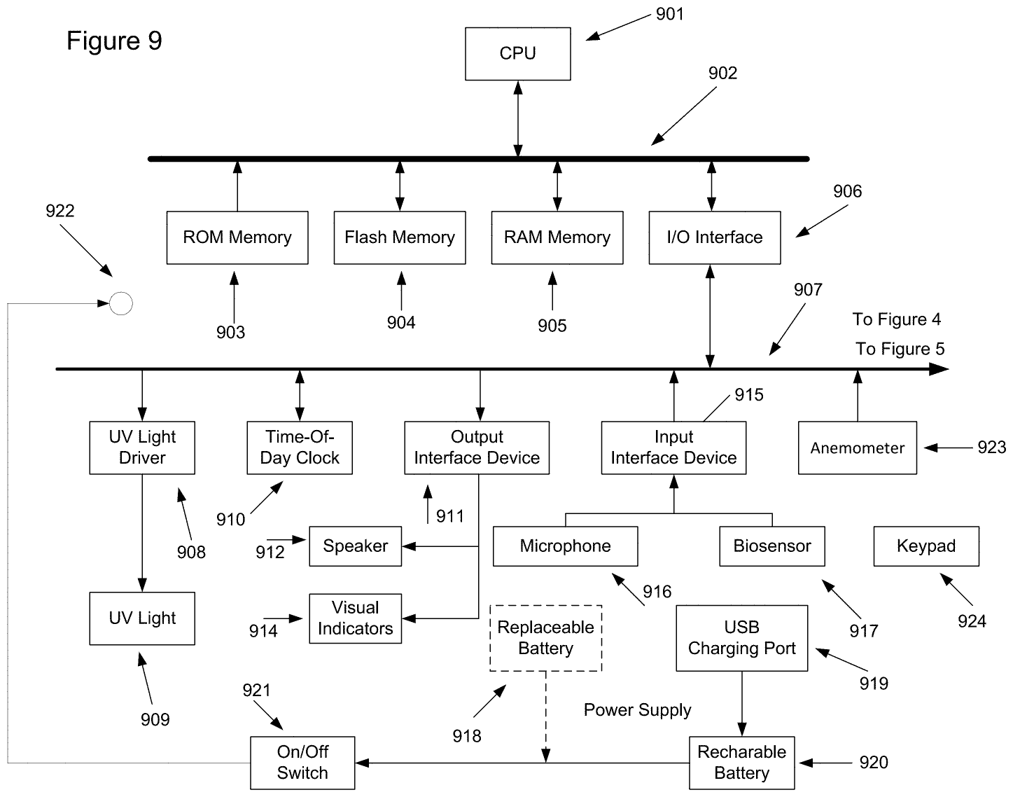

FIG. 8 illustrates an enhanced system which includes a USB Charging Port 81 for recharging Rechargeable Battery 82 in lieu of using a Replaceable Battery 83. On/Off Switch 84 supplies electrical power to the operative elements of the invention, including Anemometer 85 which measures the rate of air flow 85 into the device in response to the user breathing. The output of Anemometer 85 is a signal 87 which controls UV Light Driver 88 in order to modulate the intensity of UV Light 89 light in real time.

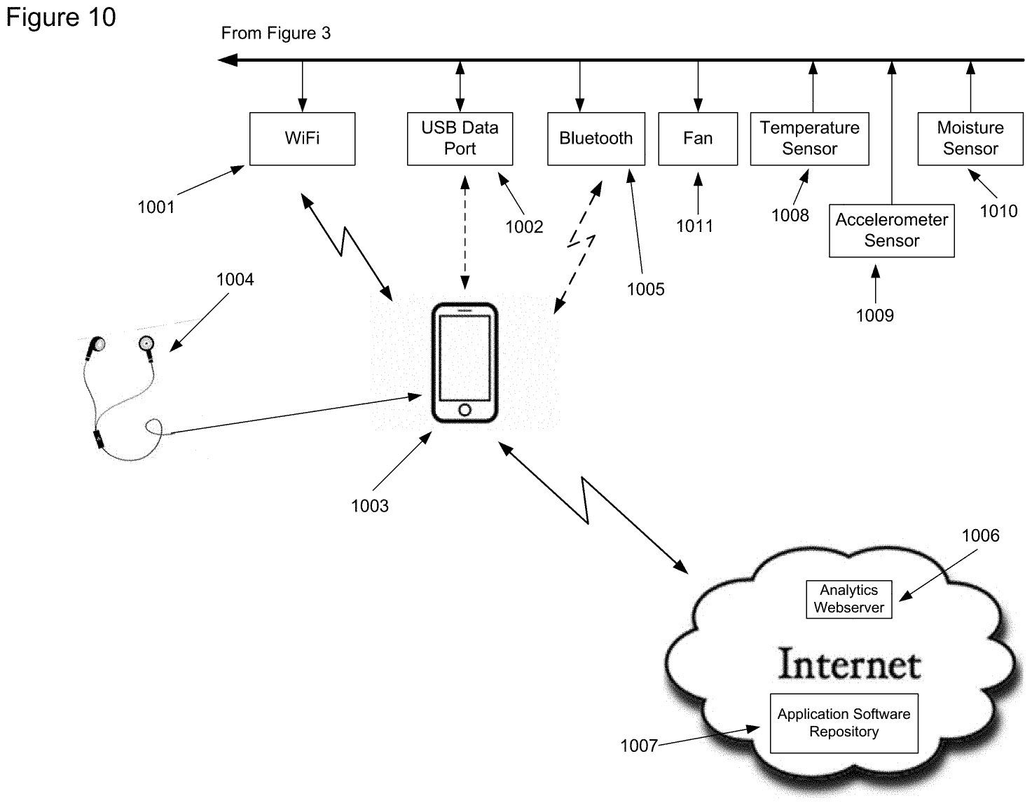

FIG. 9 is a more detailed embodiment of a control system for the present invention. In this embodiment, Central Processing Unit (CPU) 901 is provided which is used to execute computer software instructions as is known in the art. CPU 901 is coupled, via bus 902, to ROM Memory 903, Flash Memory 904, RAM Memory 905 and I/O Interface 906.
ROM Memory 903 and Flash Memory 904 may be used to store computer software instructions for execution by CPU 901.
RAM memory 905 may also be used for storing computer software instructions, and especially for storing information that is only needed for a short period of time.
I/O Interface 106 allows the system of the present invention to communicate via bus 107 to other parts of the system, such as UV Light 908, Time-Of-Day Clock 910, Output Interface Device 911, Input Interface Device 915 and Anemometer 923.

Output Interface Device 911 may be used to interface the system of the invention to the user in the form of audio alerts and messages via speaker 912 and visual alerts and messages via Visual Indicators 914. Input Interface Device 915 provides a corresponding function with respect to user inputs to the system via

Microphone 916 and Keypad 917.

As previously noted an Anemometer 923 is provided in order to monitor the air flow into the device in accordance with the user breathing.

As also previously noted, the system is power by USB Charging Port 919, Rechargeable Battery 920 and/or alternative Replaceable Battery 918 through On/Off Switch 921 to node 922. Node 911 represents the power input point to the device.

FIG. 10 is a continuation of FIG. 9 and shows connected to Buss 907 Wifi 1001, USB Data Port 1002 and Bluetooth 1005. As shown in FIG. 10, these ports can be used to connect to a smartphone 1003 and headphones 1004.

Control of the operation of Fan 1011 (which can be used to control the flow of air through the mask) as well as to take and process inputs from Temperature 1008, Accelerometer Sensor and Moisture Sensor 1010.

The system may also be directly connected to the Internet via Wifi 1001 or via smartphone 1003.

The Internet may also provide an Application Software Repository 1007 for storing a software app that can be downloaded onto smartphone 1002. Such an app would allow the smart phone to interface with the system of the present invention for the purpose of retrieving system data and providing certain control functions. Also residing on the Internet is a Analytics Server 1006. Such a server would allow data collected from a number of users of the present invention to be uploaded to the server for analytic analysis.

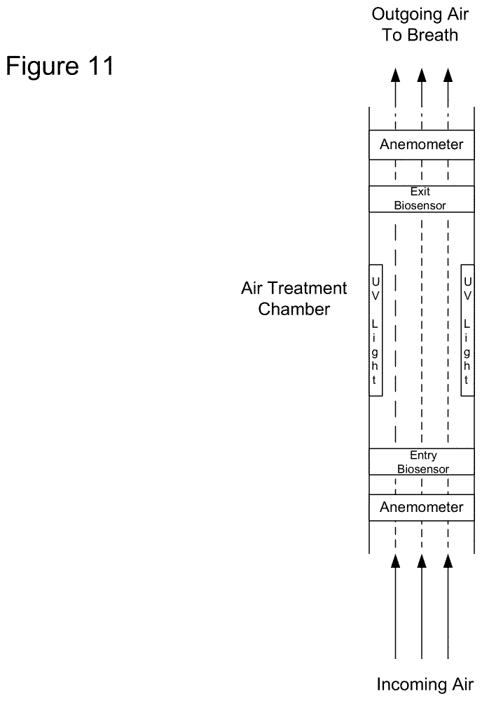

FIG. 11 illustrates the Air Treatment Chamber of the present invention where incoming air is exposed to UV Light treatment in accordance with the present invention. As shown in FIG. 11, an Entry Anemometer and an Exit Anemometer are provided for measuring air flow.

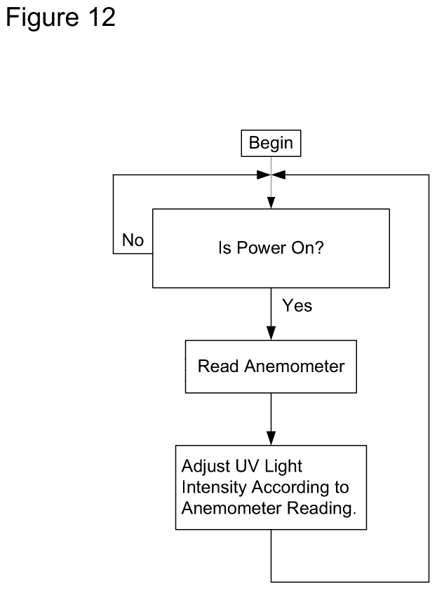

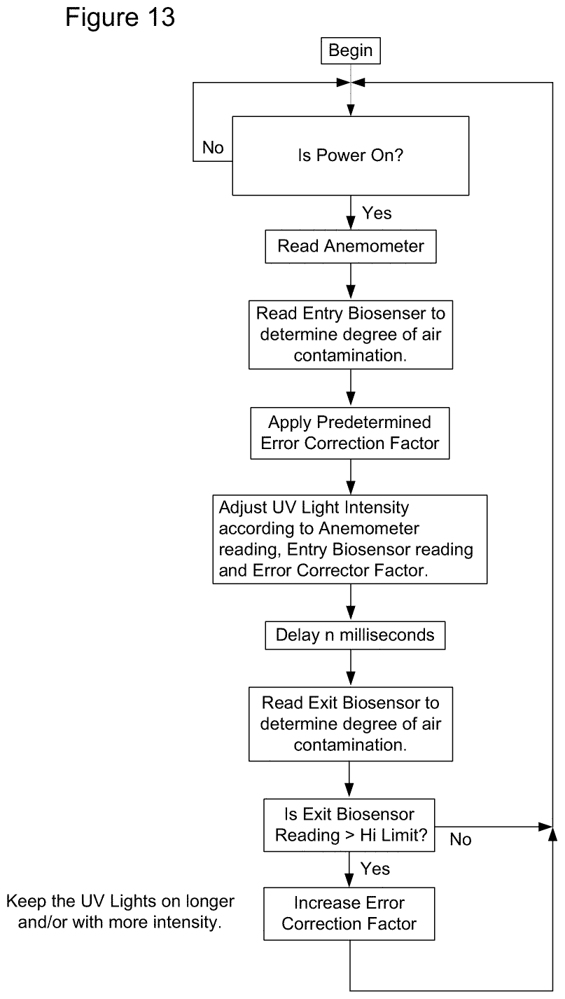

FIG. 12 is a high level flow chart of one embodiment of the operation of the present invention and FIG. 13 is a more detailed flow chart of the another embodiment of the operation.

While the foregoing specification teaches the principles of the present invention, with examples provided for the purpose of illustration, it will be appreciated by one skilled in the art from reading this disclosure that various changes in form and detail can be made without departing from the true scope of the invention.

1. A respirator mask, said mask comprising:
an air intake chamber for taking air into said mask from outside said mask;
an ultraviolet lighting system formed within the interior of said mask and having access to said air intake chamber;
a control system for controlling the operation of said lighting system, wherein said ultraviolet lighting system mitigates the health hazards caused by bacteria, germs and viruses within the air within said air intake chamber.

2. A respirator mask, said mask comprising:
a mouth piece capable of forming an airtight seal around a user's mouth;
an air chamber having an inlet port for taking air into said chamber from outside of said mask and an outlet port coupled to said mouth piece for allowing air within said chamber to pass to said mouthpiece;
an ultraviolet lighting system having access to the interior of said air chamber;
a control system coupled to said lighting system for controlling its operation in accordance with a predetermined program;
a power supply for providing electrical power to said lighting system and said control system; and
a head strap for holding said respirator mask on the head of a user.

3. The respirator mask of claim 2, wherein said lighting system mitigates the health hazards caused by bacteria, germs and viruses within said air chamber.

4. The respirator mask of claim 3, wherein said lighting system is formed of at least LED selected from the class of LEDs that emit ultraviolet type C radiation in the range of 230-280 nanometer wavelengths.

5. The respirator mask of claim 4, further including an electronic driver coupled to said LED and to said control system for controlling the intensity of said LED in response to control signals from said control system.

6. The respirator mask of claim 5, wherein said control system controls said electronic driver to drive the intensity of said LED at a predetermined intensity level.

7. The respirator mask of claim 6, wherein said intensity is based on milliwatts of said LED radiation per centimeter squared of the volume of air within said air chamber.

8. The respirator mask of claim 7, wherein said intensity is 18,000 per centimeter squared of the volume of air within said air chamber.

9. The respirator mask of claim 2, wherein said power supply is a rechargeable battery.

10. The respirator mask of claim 9, further including a USB charging port coupled to said battery for recharging said battery.

11. The respirator mask of claim 2, further including an anemometer coupled to said control system for measuring the rate of air flow through said air chamber, wherein said control system controls the intensity of said LED radiation based on the flow of air through said air chamber.

12. The respirator mask of claim 2, further including a temperature sensor coupled to said control system for measuring the temperature inside of said air chamber.

13. The respirator mask of claim 2, further including a moisture sensor coupled to said control system for measuring the amount of moisture inside of said air chamber.

14. The respirator mask of claim 2, further including a Bluetooth transceiver coupled to the said control system for communicating the operating status of said respirator mask to a remote device.

15. The respirator mask of claim 14, wherein said remote device is a smart phone.

16. The respirator mask of claim 2, further including a WiFi transceiver coupled to the said control system for communicating the operating status of said respirator mask to a remote device.

17. The respirator mask of claim 16, wherein said remote device is a smart phone.
